(19) United States
(12) Patent Application Publication
(10) Pub. No.: US 2020/0147282 A1
(43) Pub. Date: May 14, 2020

(54) REDUNDANT-IMPELLER ARTIFICIAL HEART

(71) Applicants: Peter DeSilva, (US); Steve Smith, (US)

(72) Inventor: Peter DeSilva, Rancho Santa Margarita, CA (US)

(21) Appl. No.: 16/667,695

(22) Filed: Oct. 29, 2019

Related U.S. Application Data
(63) Continuation-in-part of application No. 15/405,183, filed on Jan. 12, 2017, now Pat. No. 10,589,010.

(51) Int. Cl. A61M 1/10

(52) U.S. Cl. CPC A61M 1/1012; A61M 1/1036

(57) ABSTRACT

An artificial heart for use in a human recipient includes a housing within which a trio of turbine pump segments are operative. Two of the turbine pumps form a series connected pair for redundancy. The redundancy enhances the safety factor provided by the artificial heart. A controller is powered by a rechargeable battery and is operative to apply appropriate drive signals to the motor drives of the turbine pump segments. The battery may be implanted along with the controller to avoid the need for any external connections to the artificial heart. An inductively coupled battery charger for use outside the recipient's body is positioned proximate the battery charger to provide inductively coupled charging for use in driving the artificial heart.

### CROSS-REFERENCE TO RELATED APPLICATION

This application is a Continuation-In-Part of copending patent application Ser. No. 15/405,183 entitled REDUNDANT-IMPELLER ARTIFICIAL HEART, filed Jan. 12, 2017 in the names of Peter DeSilva and Steve Smith, the disclosure of which is incorporated herein by reference.

### FIELD OF THE INVENTION

This invention relates generally to apparatus for sustaining and continuing life for patients having failing or failed hearts and particularly to artificial heart replacement devices used therein. This invention also further relates to United States patent U.S. Pat. No. 9,314,559, issued to Steve Smith and Peter DeSilva, entitled FOUR CHAMBER REDUNDANT-IMPELLER ARTIFICIAL HEART, the disclosure of which is hereby incorporated herein by reference.

### BACKGROUND OF THE INVENTION

For many years, practitioners in the medical treatment and medical device arts have endeavored to provide artificial heart devices constructed to replace a failed or failing heart within a recipient. The most basic need is the creation of a replacement pumping device which is capable of performing the basic blood pumping and circulation functions of the natural heart.

Early attempts to provide a sustainable heart replacement were severely limited by the available technologies and the state of the art at that time. Devices proved to be generally too large and unwieldy and, for the most part, impractical. With the continuing advances in the related technologies and creative arts, heart replacement devices became smaller, more reliable and, in some instances, at least partially implantable within the recipient. Such “implantable” devices have generally remained hybrid devices in that the actual pump may be implanted within the recipient while additional support apparatus remains external to the patient and remains connected to the implanted device by a plurality of connecting wires and hoses.

Because of the complexity of human body systems and the complications and consequences of heart replacement device failure, the requirements for an implantable artificial heart remain daunting. Since the long term objectives of practitioners in the medical arts include a practical implantable artificial heart that a recipient may rely upon for long periods of life independent and free of medical supervision, reliability becomes of paramount importance.

Accordingly, and by way of example, a successful artificial heart replacement device must, above all, be long lasting and reliable. The dire consequences to the device recipient brought about by device failure make this requirement all too apparent. In addition, however, the device must be small enough to be implantable within the recipient's chest and efficient enough to maintain adequate blood circulation to sustain normal life functions. The device must avoid undue stress upon the recipient's circulatory and pulmonary systems. The device must also be capable of adjusting to and compensating for different recipient activity levels and stresses. Additional requirements such as avoidance of blood cell damage by the pumping apparatus and the prevention of the blood clot forming stagnation regions make further demands upon the heart replacement device.

In addition, because such artificial heart devices are implanted within the human recipient's chest cavity, it is essential that the size, shape and orientation of the artificial heart device the conducive to the confines of the recipient's body. Accordingly, it is an important aspect of the acceptability and practical utility of such artificial heart devices that the device minimize the intrusive potential of the device implant.

One such artificial heart device which embodies great promise, is shown in the above-referenced and incorporated U.S. Pat. No. 9,314,559 which sets forth an artificial heart for use in a human recipient that includes a housing within which a quartet of turbine pump segments are operative. The quartet of turbine pump segments provides a redundancy which in turn enhances the safety factor provided by the artificial heart. A controller is powered by a rechargeable battery and is operative to apply appropriate drive signals to the motor drives of the turbine pump segments. The battery may be implanted along with the controller to avoid the need for any external connections to the artificial heart. An inductively coupled battery charger for use outside the recipient's body is positioned proximate the battery charger to provide inductively coupled charging for use in driving the artificial heart.

While practitioners in the medical treatment and medical device arts have created a virtually endless number of proposed artificial heart replacement devices, there remains nonetheless a continuing unresolved need in the art for an improved, implantable, reliable and effective artificial heart replacement device which meets the stringent, unforgiving and vital requirements and challenges posed by a truly fully functioning completely implantable heart replacement device.

### SUMMARY OF THE INVENTION

Accordingly, it is a general object of the present invention to provide an artificial heart replacement device which is reliable, implantable and effective. It is a more particular object of the present invention to provide an artificial heart the is sized and shaped to readily fit within the interior of a human chest cavity and abdomen. It is a more particular object of the present invention to provide an improved artificial heart replacement device which avoids the need for external component apparatus and which signals events or anomalies within the system while shifting to backup remedial life sustaining operation.

In accordance with the present invention, there is provided an artificial heart comprising: a housing defining a first blood flow channel having a first input, a first output, a first turbine pump operative to flow blood from the first input to the first output; a second turbine pump operative to flow blood from the first input to the first output; a second blood flow channel having a second input, a second output, a third turbine pump operative to flow blood from the second input to the second output.

The present invention improves the art by providing an artificial heart comprising: a housing having a first blood flow channel having a first input connector and a first output connector; a second blood flow channel having a second input connector and a second output connector; a first turbine pump, having a first pump input coupled to the first input connector, and having a first pump output, the first turbine pump operative to flow blood from the first input connector to the first output connector; a second turbine pump, having a second pump input, and having a second pump output coupled to the first output connector, the second turbine pump operative to flow blood from the first input connector to the first output connector; a third turbine pump, having a third pump input coupled to the second input connector, and having a third pump output coupled to the second output connector, the third turbine pump operative to flow blood from the second input connector to the second output connector, a first blood flow passage coupling the first pump output to the second pump input.

The use of dual pump drives and dual turbine pumps within the first blood flow channel is configured to provide pump redundancy within the first blood flow channel should either of the first or second pumps fail. In such case, the remaining operative motor/pump drives the turbine coupled thereto with sufficient capability and circulation to maintain life in the recipient until remedial intervention may be performed. The inputs to the pumps and outputs from the pumps support sensors coupled to a dual microprocessor drive controller. Each microprocessor drive controller is operatively coupled to both of the redundant pump drive motors. Sensors are also provided to monitor the operation of each pump system. A pair of battery modules each including an inductively coupled charging device are implanted within the patient abdomen and operatively coupled to the processor controller and the drive motors. A pair of inductive battery charging modules are supported upon an abdominal belt and coupled to a source of operative electrical power. Battery charging is accomplished by inductive coupling through the body tissue between the external charging modules and the implanted battery and charger apparatus. The dual redundant micro controller is also implanted within the recipient's body. In a preferred fabrication of the present invention artificial heart, the first and second turbine pumps are arranged in a series pair within the first blood flow.

### BRIEF DESCRIPTION OF THE DRAWINGS

The features of the present invention, which are believed to be novel, are set forth with particularity in the appended claims. The invention, together with further objects and advantages thereof, may best be understood by reference to the following description taken in conjunction with the accompanying drawings, in the several figures of which like reference numerals identify like elements and in which:

FIG. 1 sets forth a perspective view of the present invention redundant-impeller artificial heart having the outer housing removed to show the interior components;

FIG. 2 sets forth a front view of the present invention redundant-impeller artificial heart showing the arrangement of input and output connectors upon a common surface;

FIG. 3 sets forth a perspective assembly view of the present invention redundant-impeller artificial heart showing the assembly of and illustrative turbine pump segment;

FIG. 4 sets forth a section view of the present invention redundant-impeller artificial heart taken along section lines 4-4 in FIG. 2;

FIG. 5 sets forth a section view of the present invention redundant-impeller artificial heart taken along section lines 5-5 in FIG. 2;

FIG. 6 sets forth a perspective view of the present invention redundant-impeller artificial heart together with the appropriate blood flow connections to be utilized within a human recipient;

FIG. 7 sets forth a perspective view of the present invention redundant-impeller artificial heart to gather with a support system therefore;

FIG. 8 sets forth a perspective view of an alternate embodiment of the present invention artificial heart;

FIG. 9 sets forth an end view of the alternate embodiment of the present invention shown in FIG. 8;

FIG. 10 sets forth a front view of the alternate embodiment of the present invention shown in FIG. 8;

FIG. 11 sets forth a partial assembly section view of a representative pump turbine section utilized in the alternate embodiment of the present invention alternate embodiment taken along section lines 11-11 in FIG. 9;

FIG. 12 sets forth a simplified section view of the alternate embodiment of the present invention shown in FIG. 8 showing couplings to a recipient's circulatory and pulmonary systems; and

FIG. 13 sets forth a schematic layout of the major components of the battery-power and charging apparatus of the present invention dual-stage redundant-impeller artificial heart.

### DESCRIPTION OF THE PREFERRED EMBODIMENTS OF THE INVENTION

By way of overview, the use of dual pump drives and dual turbine pumps in the present invention artificial heart within one blood flow channel is configured to provide pump redundancy should a pump fail. In such case, the remaining operative motor/pump drives the turbine coupled thereto with sufficient capability and circulation to maintain life in the recipient until remedial intervention may be performed. The inputs to the pumps and outputs from the pumps support sensors coupled to a dual microprocessor'drive controller. Each microprocessor drive controller is operatively coupled to both the redundant pump drive motors and the single pump drive motor. Sensors are also provided to monitor the operation of each pump system. A pair of battery modules each including an inductively coupled charging device are implanted within the patient abdomen and operatively coupled to the processor controller and the drive motors. A pair of inductive battery charging modules are supported upon an abdominal belt and coupled to a source of operative electrical power. Battery charging is accomplished by inductive coupling through the body tissue between the external charging modules and the implanted battery and charger apparatus.

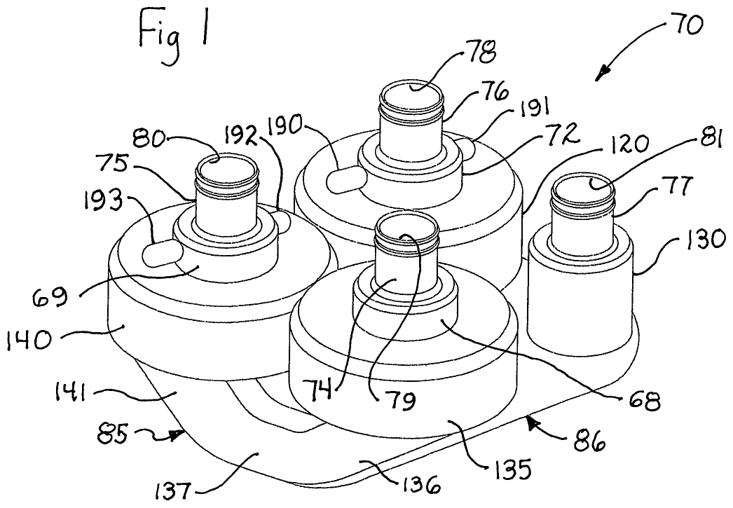

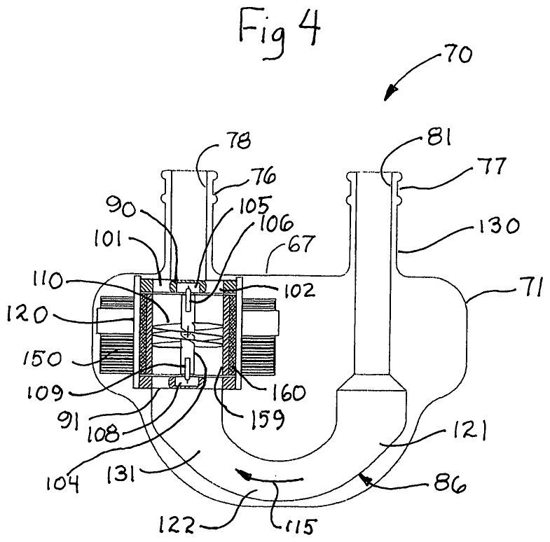

FIG. 1 sets forth a perspective view of a redundant-impeller artificial heart, generally referenced by numeral 70, having a housing 71 removed to better show the interior components of artificial heart 70. Artificial heart 70 provides an input 130, an output turbine receptacle 120. Artificial heart 70 further includes an input turbine receptacle 135 coupled to an output turbine receptacle 140 by a coupling passage 85. Coupling passage 85 includes a narrowing portion 136, a venturi portion 137 and an expanding portion 141. Similarly, as is better seen in FIG. 4 artificial heart 70 further includes a coupling passage 86 between input 130 and output turbine receptacle 120 which, in turn, supports output connector 76. Input 130 supports an output connector 77. Input turbine receptacle 135 supports an input end cap 68 which, in turn, supports and input connector 74. An output turbine receptacle 140 supports an output end cap 69 which, in turn, supports an output connector 75. Output end cap 72 further supports a pair of pressure sensors 190 and 191 similarly, output end cap 69 supports a pair of pressure sensors 192 and 193.

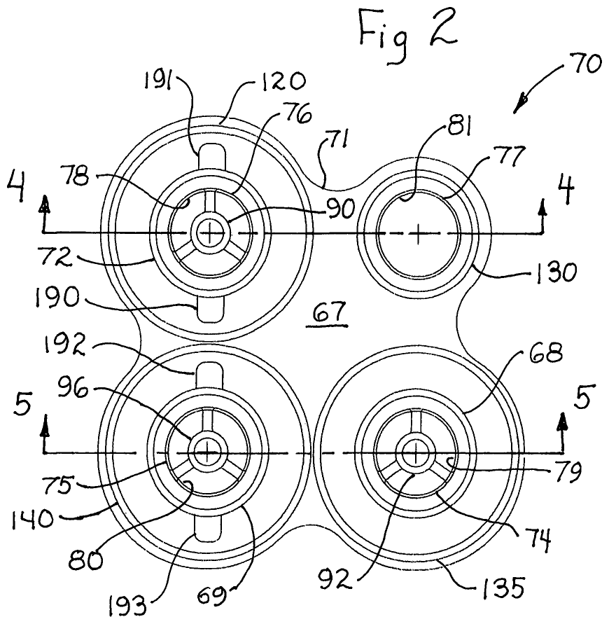

FIG. 2 sets forth a front view of redundant-impeller artificial heart 70. Artificial heart 70 is fabricated a medically approved plastic, or other implantable material. Housing 71 encloses the pump and blood flow apparatus, described below, in a smooth form fitting enclosure. Thus, within housing 71, a pair of turbine pump stages operate in one blood flow channel and utilize the redundancy provided by such multiple turbine pump stages to dramatically improve reliability. In the second blood flow channel a single turbine pump is operative. In addition, as will be seen in the figures and descriptions which follow, artificial heart 70 utilizes a “flow through” design which further improves the blood flow through the pump stages and the blood flow between the pump stages to provide increased efficiency and a further protection against the stagnation or pooling of blood within the artificial heart. The latter is extremely important in that blood which is allowed to pool or stagnate within the artificial heart raises the potential for injurious or even fatal clotting of blood within the recipient's circulatory system. To avoid such problems, artificial heart 70 utilizes a direct flow through configuration through curved, preferably, venturi passages which increases blood flow velocity and does not provide areas of potential of blood stagnation or blood pooling.

Accordingly, as set forth above, artificial heart 70 having housing 71 defines a common surface 67, which in turn, supports input end cap 68 as well as output end caps 69 and 72. End cap 68 supports an input connectors 74. Similarly, output end caps 69 and 72 support output connectors 75 and 76 respectively. Input connectors 74 and 77 define passages 79 and 81 respectively while output connectors 75 and 77 define respective passages 80 and 78. End caps 68, 69 and 72 are secured to housing 71 utilizing an approved method of fabrication such as thermal or sonic welding. Alternatively, assembly to housing 71 may be secured utilizing adhesive attachments or, in some circumstances, medically approved fasteners. Of importance to note in determining the assembly of end caps 68, 69 and 72 to housing 71 is the provision of a stable, secure and reliable attachment therebetween such that the combination thus formed becomes, in essence, a single integral housing supporting the internal turbine pumps (shown below). Turbine supports 90, 92, and 96 are also seen through passages 78, 79, and 80 respectively.

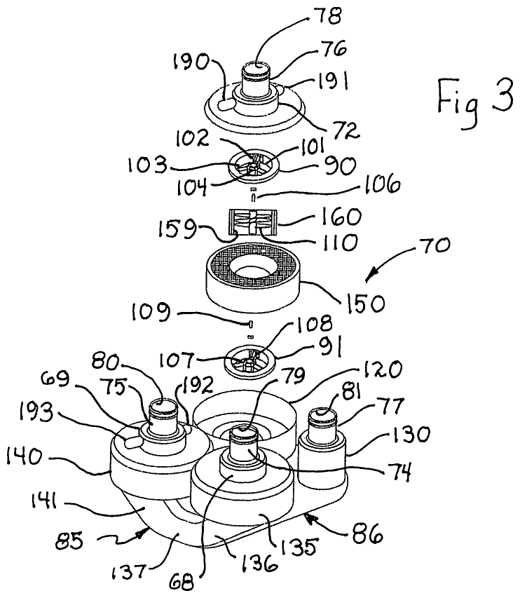

FIG. 3 sets forth a partial perspective partial assembly view of redundant-impeller artificial heart 70 having housing 71 removed to expose the artificial heart interior components. It will also be noted that FIG. 3 shows an assembly view of an illustrative turbine pump stage operable within artificial heart 70. It will be understood that the remaining turbine pump stages within artificial heart 70 are substantially identical. Accordingly, the illustrations and descriptions in the assembly view portion set forth in FIG. 3 will be understood to be equally descriptive of and equally applicable to the remaining turbine pump stages within artificial heart 70.

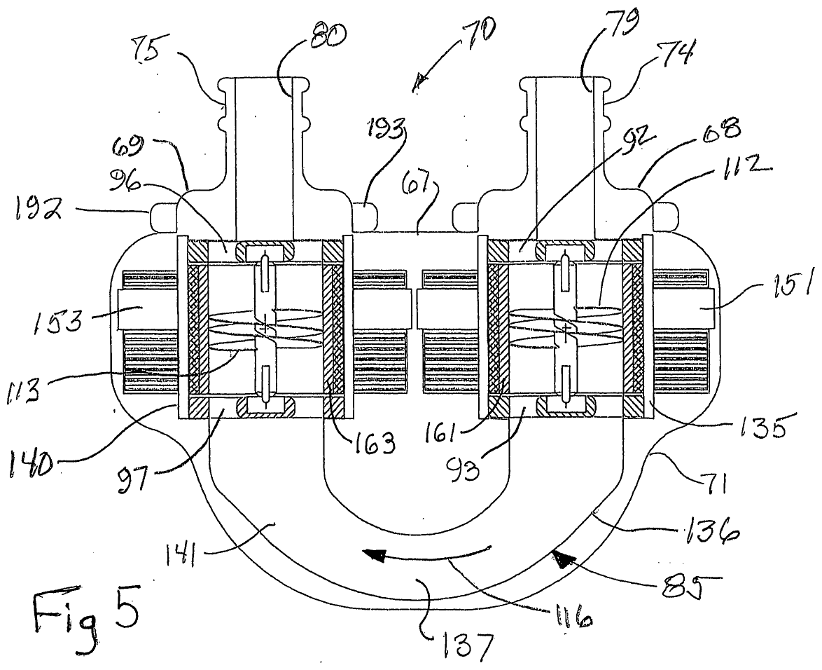

More specifically, as is best seen in FIG. 5, artificial heart 70 includes an input turbine receptacle 135 and an output turbine receptacle 140. Input turbine receptacle 135 is coupled to output turbine receptacle 140 by a coupling passage 85. Coupling passage 85 includes a narrowing portion 136, a venturi portion 137 and an expanding portion 141. The combination of input turbine receptacle 135, coupling passage 85 and output turbine receptacle 140 forms a blood flow channel from input 74 to output 75.

Similarly, as is better seen in FIG. 4, artificial heart 70 further includes an input 130 and a turbine output receptacle 120. A coupling passage 86 formed of a narrowing version 121, a venturi portion 122 and an expanding portion 141 couples input 130 to turbine output receptacle 120. The combination of input 130, coupling passage 86 and turbine output receptacle 120 forms a blood flow channel from input 77 to output 76.

Onput turbine receptacle 120 supports an input end cap 72 which in turn supports output connector 76. Input turbine receptacle 135 supports an input end cap 68 which, in turn, supports and input connector 74. An output turbine receptacle 140 supports an output end cap 69 which, in turn, reports and output connector 75. Output end cap 72 further supports a pair of pressure flow sensors 190 and 191 similarly, output end cap 69 supports a pair of pressure sensors 192 and 193.

With reference to the assembly view portion of FIG. 3, turbine 110 is preferably fabricated to provide a helical blade progressive to form a helix. Turbine 110 further supports a cylindrical magnetic rotor 160 joined to a cylindrical metal sleeve 159 which is joined to the outer edges of turbine 110. Magnetic rotor 160 supports a plurality of permanent magnets and together with turbine 110 forms a single preferably integrally fabricated rotating component. Thus, for example, it will be recognized that while turbine 110 may be precision-fitted within sleeve 159 of magnetic rotor 160 due to the cylindrical structure of magnetic rotor 160 to form a single rotating unit, in the preferred fabrication of the present invention magnetic rotor 160 is integrally formed and molded with turbine 110. In either event, it will be recognized that the combined structure of turbine 110 and magnetic rotor 160 forms a single integral rotating unit. The combined structure of magnetic rotor 160 and turbine 110 are rotatably supported within the interior of turbine receptacle 120 by a pair of bearing supports 90 and 91 positioned on each side of the rotating turbine element. The structure of bearing supports 90 and 91 is set forth below in FIG. 4. Thus, bearing support 90 includes a center hub 104 supported by a plurality of spokes 101, 102 and 103. Within hub 104, a bearing cup 105 is supported which in turn receives one end of a bearing pin 106.

Bearing support 91 is identical to bearing support 90 and thus includes a center hub 107 which receives a bearing cup 108 and bearing pin 109. During assembly, bearing support 91 receives bearing cup 108 and is inserted in turbine receptacle 120 formed in housing 71. Thereafter, bearing pins 106 and 109 are inserted into the support shaft of turbine 110. The combined structure of turbine 110 supporting bearing pins 106 and 109 together with magnetic rotor 160 is then inserted into turbine receptacle 120. Bearing support 90 is then fitted within turbine receptacle 120 such that bearing pin 160 is received within bearing cup 105. The remaining turbine segments are each assembled within their respective turbine receptacles into housing 71. Once the turbine and magnetic rotor combination have been assembled within housing 71, end caps 68, 69 and 72 are joined to their respective turbine receptacles. using an attachment such as thermal or sonic welding or other appropriate attachment. Once the end caps are assembled to, the structure of artificial heart 70 is complete and the resulting pump structure may be described.

FIGS. 4 and 5 set forth section views of artificial heart 70 taken along section lines 4-4 and 5-5, respectively in FIG. 2. The section views shown in FIGS. 4 and 5 show each of the parallel pumping apparatus forming artificial heart 70. As is set forth above, the pumping apparatus includes curved venturi coupling passages. It will be understood that, in the preferred fabrication of the present invention, the three turbine pumps (turbines 110,112 and 113) are identical. It will be noted that two isolated blood flow channels are formed within artificial heart 70. Thus, separate blood flow channels are formed, one between input 77 and output 76 and the other between input 74 and output 75.

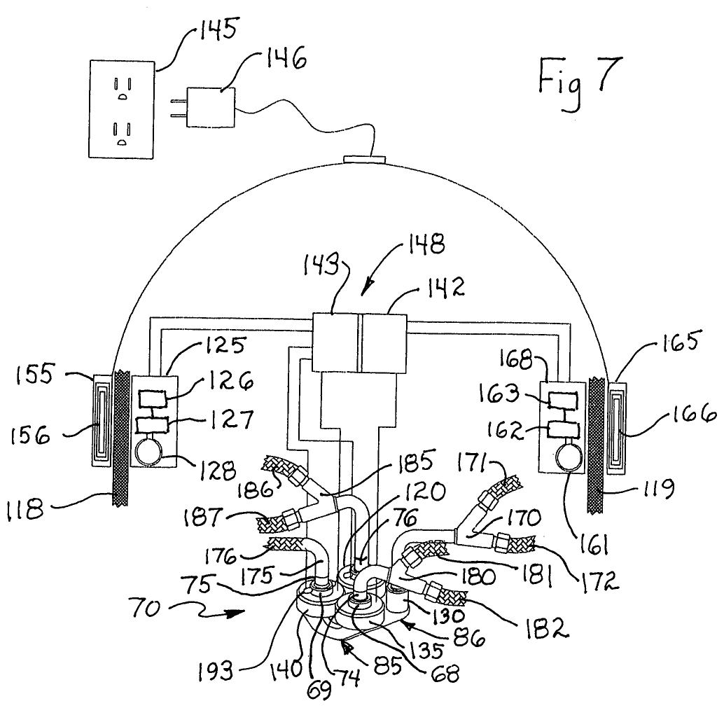

Accordingly and with concurrent reference to FIGS. 4 and 5, the interior structure of artificial heart may be described. Housing 71 further supports a generally cylindrical drive coil array 150 which encircles turbine receptacle 120. Drive coil assembly 150 provides a motor drive coil which is supported within housing 71. Drive coil 150 is coupled to a motor controller such as controller 148 set forth above in FIG. 7. Drive coils 150, 151, and 153 are substantially identical in fabrication.

Artificial heart 70 includes a turbine 110 and magnetic rotor 160 joined to form a single rotating structure which is rotatably supported within turbine receptacle 120 by bearing supports 90 and 91. Artificial heart 70 further includes a turbine 112 and magnetic rotor 161 also joined to form a single rotating structure which is rotatably supported within turbine receptacle 135 by bearing supports 92 and 93. Finally, artificial heart 70 further includes turbine 113 and magnetic rotor 163 joined to form a single rotating structure which is rotatably supported within turbine receptacle 140 by bearing supports 96 and 97.

As mentioned above, artificial heart 70 utilizes three turbine pump stages two of which (112 and 113) are arranged in a series coupled pair with the remaining turbine pump stage (110) operating singularly. It will be equally well appreciated that each of the three pump stages operative within turbine receptacles 120, 135 and 140 includes a drive coil supported within housing 171 and a rotating rotor formed by the combination of a turbine and a magnetic rotor. The resulting combinations are often referred to in the art as “frameless servo motors”. However, it will be apparent to those skilled in the art that other motor drive structures may be used to rotate the turbines without departing from the spirit and scope of the present invention. In accordance with an important aspect of the present invention, it will be noted that each of the three pump stages may be independently operated and controlled as to speed and output. It will be further apparent to those skilled in the art that the use of a pair of pump stages provides a redundant pump stage arrangement that allows either pump stage to continue to provide blood flow despite a failure of either pump stage.

In operation, the three pump stages of artificial heart 70 are driven in a manner described below by drive and control apparatus shown in FIG. 7. Accordingly, appropriate electrical signals are applied to drive coils 150, 151, and 153 to induce rotation of magnetic rotors 160, 161, and 163 which produces rotation of the rotatably supported turbines 110 112, and 113 along with their respective magnetic rotors 160, 161, and 163. As is described below in greater detail, it will be noted that the rotations of turbine 110 produces a straight through curved flow path in the direction indicated by arrow 115 between input 77 and output 76. This straight through curved flow path is enhanced by the venturi coupling between turbine receptacles 120 and 130 provided by narrowing portion 121, venturi portion 122 and expanding portion 131 of curved coupling passage 86. The purpose of the venturi coupling is to increase the flow velocity between the pump turbines and further enhance the blood flow between input 76 and output 77. As a result of the straight-through blood flow thus produced, areas of stagnation and blood pooling are avoided.

In a similar fashion and for similar reasons, the blood flow from input 74 to output 75 between turbines 112 and 113 is similarly enhanced by the venturi coupling therebetween provided by narrowing portion 136, venturi portion 137 and expanding portion 141 of coupling passage 85. Once again, a direct flow-through blood flow path between input 74 and output 75 is provided. This flow path in the direction indicated by arrow 116 is enhanced by the increased flow velocity created by the venturi coupling and avoids stagnation and blood pooling.

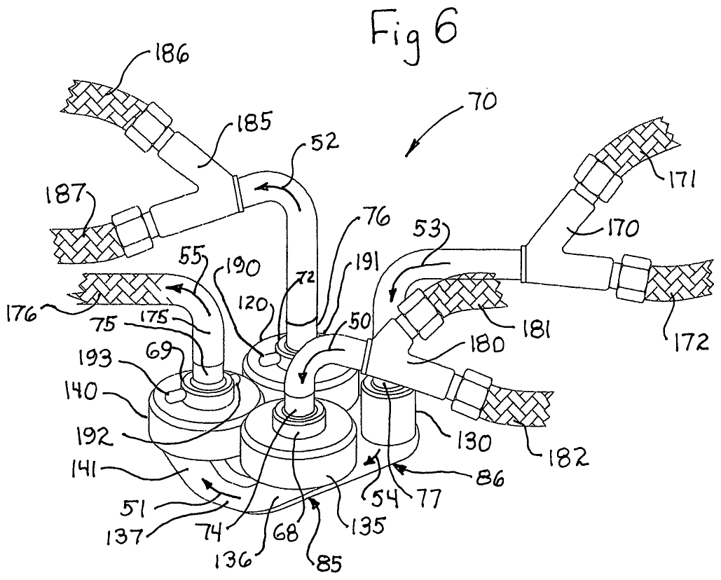

FIG. 6 sets forth a perspective view of artificial heart 70 showing appropriate connections to the recipient's circulatory system. The blood flow connections are provided by medically approved tube elements and couplers. Artificial heart 70 includes a housing 71 described above and shown in FIGS. 4 and 5.

In FIG. 6, artificial heart 70 is shown having housing 71 removed to better show the interior components of artificial heart 70. Artificial heart 70 provides an input turbine receptacle 120 and a turbine output 130. Artificial heart 70 further includes an input turbine receptacle 135 and an output turbine receptacle 140. Input turbine receptacle 135 is coupled to output turbine receptacle 140 by a coupling passage 85. Coupling passage 85 includes a narrowing portion 136, a venturi portion 137 and an expanding portion 141. Similarly, as is better seen in FIG. 4 artificial heart 70 further includes a coupling passage 86 formed of a narrowing version 121, a venturi portion 122 and an expanding portion 131. Output turbine receptacle 120 supports an output end cap 72 which in turn supports output connector 76. Turbine input 130 supports an input connector 77. Input turbine receptacle 135 supports an input end cap 68 which, in turn, supports and input connector 74. An output turbine receptacle 140 supports an output end cap 69 which, in turn, supports an output connector 75. Output end cap 72 further supports a pair of pressure sensors 190 and 191. Similarly, output end cap 69 supports a pair of pressure sensors 192 and 193.

Input 130 of pump 70 is coupled to a split coupler 170 which in turn is coupled to the recipient's superior vena cava by a tube 171 and is further coupled to the recipient's inferior vena cava by a tube 172. Similarly, output 76 of turbine output receptacle 120 is coupled to a split coupler 185 which is coupled to the recipient's pulmonary arteries by a pair of tubes 186 and 187. In addition, input 74 is coupled to a split coupler 180 which is coupled to the recipient's pulmonary veins by pair of tubes 181 and 182. Finally, output connector 75 is coupled to a tube 176 by a coupler 175. Tube 176 is coupled to the recipient's aorta. Output connector 76 further supports a pair of output transducers 190 and 191 while output 75 supports a pair of output transducers 192 and 193. Transducers 190, 191, 192 and 193 are coupled to the artificial heart controller in the manner set forth below in FIG. 7.

In operation, as artificial heart 70 is operated, blood is drawn through input 130 into pump 70 from the patient's superior and inferior vena cava through split coupler 170 and tubes 171 and 172 in the direction indicated by arrow 53. This blood is pumped through coupling passage 86 in the direction indicated by arrow 54, also seen as arrow 115 in FIG. 4, and is further pumped in the direction indicated by arrow 52 outwardly through output connector 76 and thereafter passes through split connector 185 and tubes 186 and 187 to the recipient's pulmonary arteries. The blood flows through the patient's lungs and returns to artificial heart 70 through tubes 181 and 182 and split coupler 180 in the direction indicated by arrow 50 through input 74. Blood is then pumped through coupling passage 85 in the direction indicated by arrow 51 and is further pumped passing outwardly through output 75 and coupler 175 in the direction indicated by arrow 55 to be carried by tube 176 to the recipient's aorta for distribution throughout the recipient's circulatory system.

Sensors 190 and 191 monitor the output pressure at output 76. Similarly, transducers 192 and 193 monitor the output pressure at output 75. In response to variations of sensed pressure at either of outputs 75 or 77, the system controller (seen in FIG. 7) is able to adjust the operating turbine segments of the artificial heart to compensate for any failures within the turbines. In addition, it will be apparent to those skilled in the art that the drive signals applied to drive the turbine pumps may also be modulated or varied to provide a pulsitile output pressure from either or both of outputs 75 and 77. This pulsitile character imparts a more natural blood flow pattern for the recipient and may prove to be advantageous to the recipient.

FIG. 7 sets forth a block diagram of the present invention artificial heart in a typical surrounding environment. In the situation represented in FIG. 7, artificial heart 70 has been implanted within a host patient's body and is operatively coupled in the manner set forth above in FIG. 6 to the host patient's circulatory system. FIG. 7 further shows a microcontroller unit 148 also implanted within the host patient's body. Microcontroller unit 148 is formed of a pair of fully redundant micro controllers 143 and 142. The redundancy of micro controllers 143 and 142, each able to fully support the operation of artificial heart 70 provides a further measure of reliability. Microcontroller unit 140 further includes conventional apparatus (not shown) for communicating to the exterior of the host patient's body in order to provide alarm condition information or other required maintenance of monitoring information to an external unit (not shown). As described above, artificial heart 70 includes a plurality of sensors 190, 191, 192 and 193 situated at the respective inputs and outputs of artificial heart 70. Sensors 190 through 193 are coupled to redundant microcontrollers 143 and 142. Microcontroller 142 further includes additional sensors supported within artificial heart 70 for monitoring the performance of the servo drive apparatus therein. A pair of battery units 125 and 168 are also implanted within the host patient. Battery unit 125 includes a secondary charging coil 128 coupled to a rectifier 127 which in turn is coupled to a battery 126. Battery unit 125 is coupled to microcontroller 143. Similarly, battery unit 168 includes a charging coil 161 coupled to a rectifier 162 which in turn is coupled to a battery 163. By way of further similarity, battery unit 168 is operatively coupled to microcontroller 142. Thus, microcontroller unit 140, artificial heart 70 and battery units 125 and 168 together with appropriate wire connections therebetween are implanted within a host patient body. For purpose of illustration, FIG. 7 shows body segments 118 and 119 which represent the skin and associated tissues of the host patient body beneath which battery units 125 and 168 are implanted. Preferably units 125 and 168 are implanted near the host patient's mid section and preferably situated just beneath the patient's skin.

A charging belt 158 suitably configured to be worn by the host patient such as at or near the patient's waist supports a pair of charging units 155 and 156. Charging units 155 and 165 include respective primary charging coils 156 and 166. Coils 156 and 166 are coupled to source of alternating current power such as a conventional electrical outlet 145 via a conventional coupling adapter 146.

In operation, micro controller 143 and 142 monitor the plurality of sensors within artificial heart 70 and provide suitable operating power and control to the servo drives supported therein (seen in FIG. 5). Microcontrollers 143 and 142 utilize batteries 126 and 163 for operative battery supply and for power to energize the servo drive apparatus within artificial heart 70. The operative power stored within batteries 126 and 163 is provided by inductive charging utilizing charging units 155 and 165. Thus, during convenient periods, the host patient utilizes charging belt 158 by coupling to power source 145 while wearing belt 158 such that primary charging coils 156 and 166 are positioned on the outside of body portions 118 and 119 respectively such that general alignment is obtained between primary charging coils 156 and 166 and secondary coils 151 and 161 respectively. Electrical power is then inductively coupled through body portions 118 and 119 to induce alternating current power within secondary coils 151 and 161. Rectifiers 127 and 162 convert the alternating current induced in coils 128 and 161 to a direct current power suitable for charging batteries 126 and 163. In this manner, the user is able to replenish the battery energy as required by simply wearing charging belt 158 for a suitable time interval.

Micro controller unit 148 functions using a pair of fully-redundant fully-interconnected micro controllers, each having the complete capability to control and run the entire artificial heart system and it's monitoring and charging functions. Thus, micro controllers 143 and 142 provide inputs for two batteries, inputs for multiple pressure and Hall effect servo sensors and systems capable of monitoring multiple battery charge levels and switch between batteries. The redundancy of micro controllers 143 and 142 includes configuration of the system such that each micro controller “sees” all its own inputs and also “sees” all inputs to the other micro controller. This redundancy includes each micro controller being capable of making compensating performance adjustments to maintain envelope system performance. However, to avoid “hunting” between the redundant micro controllers, it is preferred that small pressure variations of each pump be allowed before adjustment is made.

Micro controller unit 148 further includes communication capability, such as a wireless unit, to call, or text remote locations to indicate system anomalies, failures, operating conditions, battery charge levels and other conditions. In addition, micro controller unit 140 provides the capability to adjust each of micro controllers 143 and 142 based on pressure readings and to set and maintain preset maximum and minimum pressure envelopes.

Micro controller unit 148 also provides the ability of replicating the pulsitile operation characteristic of a normal human heart by introducing pre-programmed increases and decreases of pump speed to create pressure surges and lulls.

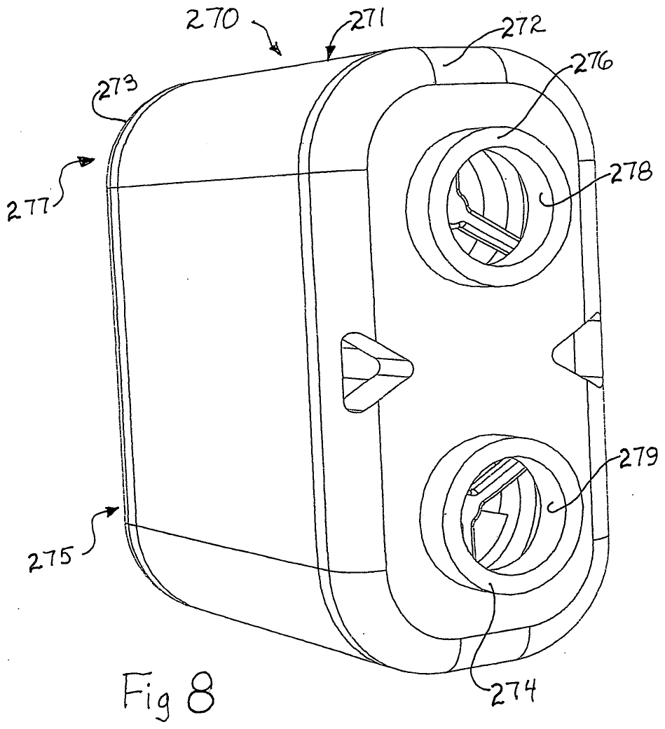

FIG. 8 sets forth a perspective view of an alternate embodiment of the present invention dual-stage redundant-impeller artificial heart constructed in accordance with the present invention and generally referenced by numeral 270. Artificial heart 270 is fabricated in general correspondence to artificial heart 70 described above and set forth in FIGS. 1 through 7. Thus, artificial heart 270 provides multiple turbine pump stages in the same general manner described above in artificial heart 70 and utilizes the redundancy provided by such multiple turbine pump stages. However, as will be seen in the figures and descriptions which follow, artificial heart 270 utilizes a “flow through” design which further improves the blood flow through the pump stages and the blood flow between the pump stages to provide increased efficiency and a further protection against the stagnation or pooling of blood within the artificial heart. This is extremely important in that blood which is allowed to pool or stagnate within the artificial heart raises the potential for injurious or even fatal clotting of blood within the recipient's circulatory system. To avoid such problems, artificial heart 270 utilizes a direct flow through configuration which does not provide areas of potential of blood stagnation or blood pooling.

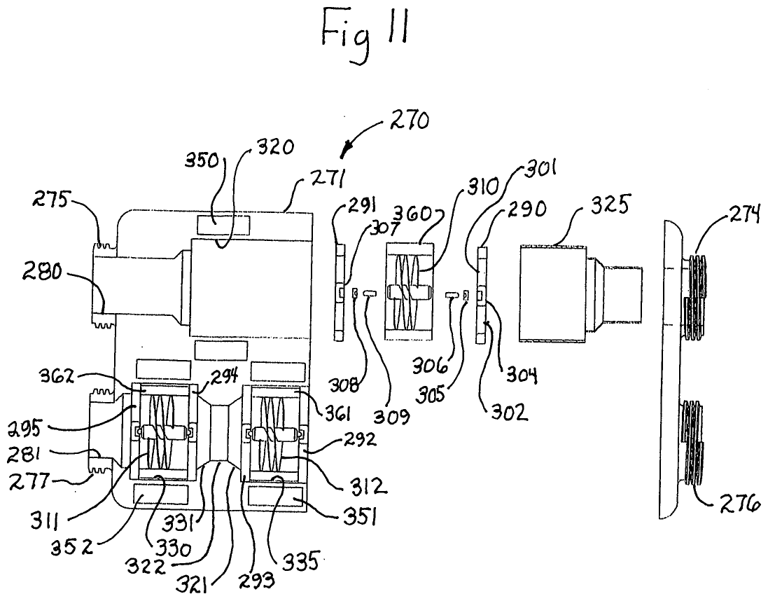

More specifically, FIG. 8 sets forth artificial heart 270 having a center housing 271 supporting a pair of manifolds 272 and 273 on opposite ends thereof. Manifold 272 includes an input coupling 276 defining an input passage 278 therein. Similarly, manifold 272 includes an input coupling 274 defining an input passage 279. As is better seen in FIG. 11, manifold 273 is constructed in a similar fashion to manifold 272 and thus defines a pair of output couplings 275 and 277 which define respective output passages 280 and 281. Returning to FIG. 8, in the preferred fabrication of artificial heart 270, manifolds 272 and 273 are fitted to center housing 271 after the internal pump components (shown below) are assembled within housing 271. Also in the preferred fabrication of the present invention, housing 271 as well as manifolds 272 and 273 are formed of a suitable medically approved plastic material. Thus, manifolds 272 and 273 are secured to housing 271 utilizing an approved method of fabrication such as thermal or sonic welding. Alternatively, assembly of manifolds 272 and 273 to housing 271 may be secured utilizing adhesive attachments or, in some circumstances, medically approved fasteners. Of importance to note in determining the assembly of manifolds 272 and 273 to housing 271 is the provision of a stable, secure and reliable attachment therebetween such that artificial heart 270 becomes, in essence, a single integral housing supporting the internal turbine pumps (shown below).

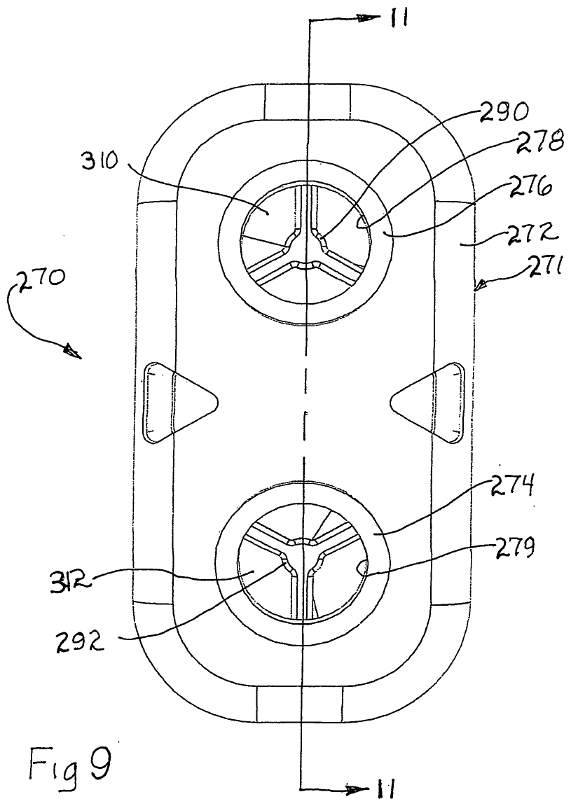

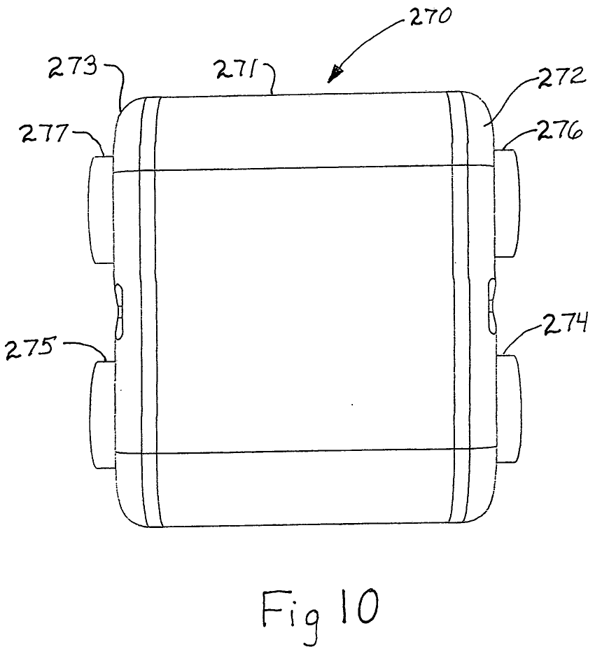

FIG. 9 sets forth an end view of artificial heart 270 showing manifold 272 secured to center housing 271 (better seen in FIG. 8). As described above, manifold 272 supports a pair of input couplers 276 and 274. As is also described above, input couplers 276 and 274 define respective input passages 278 and 279. In the end view of artificial heart 270 shown in FIG. 9, portions of bearing supports 290 and 292 together with portions of turbines 310 and 312 may be seen through input passages 278 and 279. While the structure of the turbine pump segments within artificial heart 270 is shown below in greater detail, suffice it to note here that the direct flow which occurs through input passages 278 and 279 passing through, bearing supports 290 and 292 respectively and into turbine pump sections 310 and 312. This fabrication in repeated on the opposite side of housing 271 in the configuration of manifold 273 (seen in FIG. 10). Suffice it to note here that as turbines 310 and 312 are rotated under the influence of magnetically coupled drive apparatus (seen in FIG. 11), the rotation of turbines 310 and 312 draws blood through passages 278 and 279 into the interior of artificial heart 270.

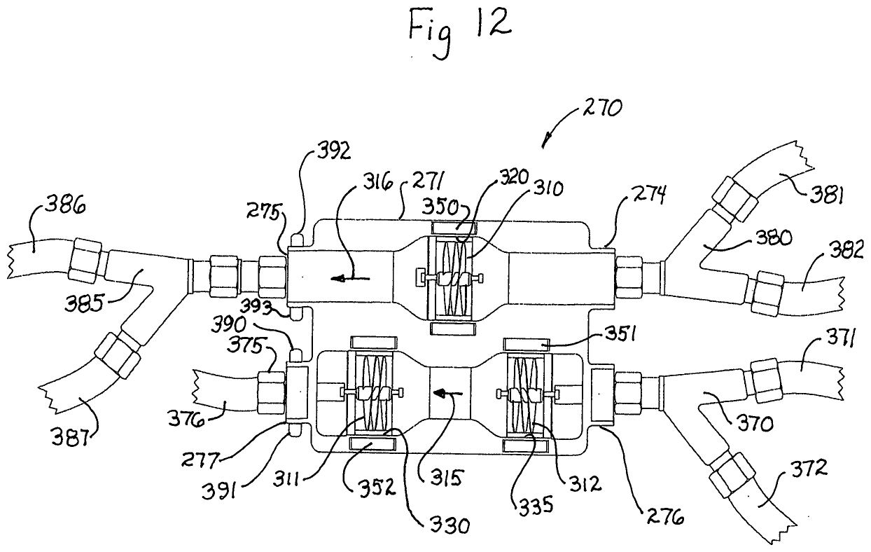

FIG. 10 sets forth a front view of artificial heart 270 showing enter housing 271 supporting manifolds 272 and 273. As described above, manifold 272 supports input couplers 276 and 274 while manifold 273 supports output couplers 277 and 275. As is better shown below in FIG. 12, couplers 276 and 274 are coupled to provide blood flow inputs to pump 270 while couplers 277 and 275 are utilized in providing blood flow outputs for pump 270.

FIG. 11 sets forth a partial section partial assembly section view of artificial heart 270. In particular, FIG. 11 shows an assembly view of the structure for turbine pump 310. It will be apparent to those skilled in the art that the structure described for pump 310 is identical to the structure utilized for turbines 311 and 312. Accordingly, the descriptions set forth herein for the supporting structure and drive apparatus for turbine 310 will be understood to apply equally well to the structure and drive apparatus of turbines 311 and 312.

More specifically, turbine 310 is preferably fabricated to provide a helical blade progressive to form a helix. Turbine 310 further supports a cylindrical magnetic rotor 360 which is joined to the outer edges of turbine 310. Magnetic rotor 360 supports a plurality of permanent magnets and together with turbine 310 forms a single preferably integrally fabricated rotating component. Thus, for example, it will be recognized that while turbine 310 may be precision-fitted within magnetic rotor 360 due to the cylindrical structure of magnetic rotor 360 to form a single rotating unit, in the preferred fabrication of the present invention magnetic rotor 360 is integrally formed and molded with turbine 310. In either event, it will be recognized that the combined structure of turbine 310 and magnetic rotor 360 forms a single integral rotating unit. The combined structure of magnetic rotor 360 and turbine 310 are rotatably supported within the interior of housing 271 by a pair of bearing supports 290 and 291 positioned on each side of the rotating turbine element. The structure of bearing supports 290 and 291 is set forth above in FIG. 3. Thus, bearing support 290 includes a center hub 304 supported by a plurality of spokes 301, 302 and 303 (spoke 303 not shown). Within hub 304, a bearing cup 305 is supported which in turn receives one end of a bearing pin 306.

Bearing support 291 is identical to bearing support 290 and thus includes a center hub 307 which receives a bearing cup 308 and bearing pin 309. During assembly, bearing support 291 receives bearing cup 308 and is inserted in turbine receptacle 320 formed in housing 271. Thereafter, bearing pins 306 and 309 are inserted into the support shaft of turbine 310. The combined structure of turbine 310 supporting bearing pins 306 and 309 together with magnetic rotor 360 is then inserted into turbine receptacle 320. Bearing support 290 is then fitted within turbine receptacle 320 such that bearing pin 360 is received within bearing cup 305. Finally, plug 325 is fitted into receptacle 320 trapping bearing support 290 and securing turbine 310 and its support structure. A suitable attachment of plug 325 to housing 271 is applied to secure the turbine pump. The remaining turbine segments are each assembled within their respective turbine receptacles into housing 271. Once the three turbine and magnetic rotor combinations have been assembled within housing 271, manifolds 272 and 273 are joined to center housing 271 using an attachment such as thermal or sonic welding or other appropriate attachment. With manifolds 272 and 273 are assembled to center housing 271, the structure of artificial heart 270 is complete and the resulting pump structure may be described.

More specifically, artificial heart 270 includes a center housing 271 defining a plurality of turbine receptacles 320, 330 and 335. Receptacles 330 and 335 are aligned coaxially and define cylindrical receptacles. Turbine receptacles 330 and 335 are coupled by a venturi coupling passage formed by a tapered portion 321, a center passage 322 and a tapered portion 331 which are also generally coaxial with turbine receptacles 335 and 330.

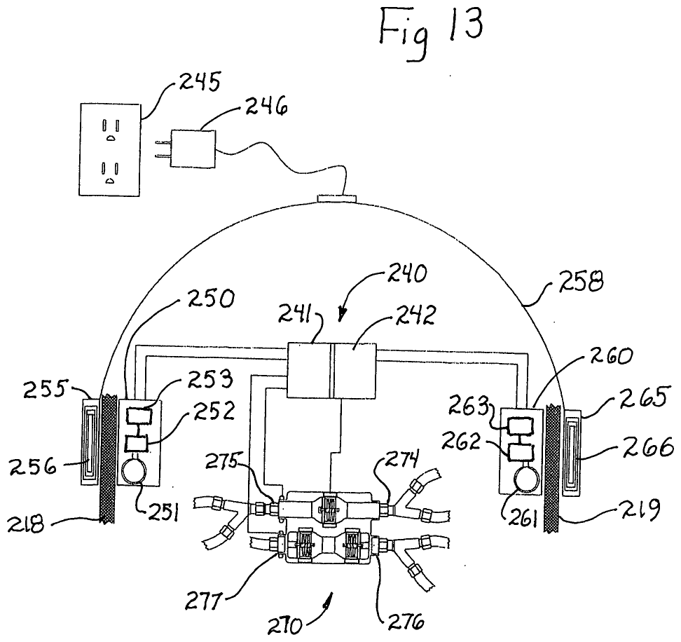

Turbine receptacle 320 is coupled to output 275 and input 274 all three of which are mutually coaxial. Housing 271 further supports a generally cylindrical drive coil array 350 which encircles turbine receptacle 320. Drive coil assembly 350 provides a motor drive coil which is molded into housing 271. Drive coil 350 is coupled to a motor controller, such as controller 240, set forth below in FIG. 13. Similarly, housing 271 supports a corresponding drive coil 352 which encircles turbine receptacle 330 and a drive coil 351 which encircles turbine receptacle 335. Drive coils 350, 351 and 352 are substantially identical in fabrication.

As described above, artificial heart 270 includes a turbine 310 and magnetic rotor 360 joined to form a single rotating structure which is rotatably supported within turbine receptacle 320 by bearing supports 290 and 291. Artificial heart 270 further includes a turbine 312 and magnetic rotor 361 also joined to form a single rotating structure which is rotatably supported within turbine receptacle 335 by bearing supports 292 and 293. Artificial heart 270 further includes turbine 311 and magnetic rotor 362 joined to form a single rotating structure which is rotatably supported within turbine receptacle 330 by bearing supports 294 and 295.

Thus, it will be appreciated that artificial heart 270 utilizes three turbine pump stages, two of which, 311 and 312, are arranged in a series coupled pair and one of which 310, operates alone. It will be equally well appreciated that each of the three pump stages operative within turbine receptacles 320, 330 and 335 includes a drive coil supported within housing 271 and a rotating rotor formed by the combination of a turbine and a magnetic rotor. The resulting combination are often referred to in the art as “frameless servo motors”. However, it will be apparent to those skilled in the art that other servo motor drive structures may be used to rotate the turbines without departing from the spirit and scope of the present invention. In accordance with an important aspect of the present invention, it will be noted that each of the three pump stages may be independently operated and controlled as to speed and output. It will be further apparent to those skilled in the art that the use of two of the pump stages in a series coupled pair provides a redundant pump stage arrangement that allows either pump stage to continue to provide blood flow despite a failure of either pump stage.

In operation, the three pump stages of artificial heart 270 are driven in a similar manner to the above-described drive and control apparatus operative in combination with artificial heart 70. Accordingly, appropriate electrical signals are applied to drive coils 350, 351 and 352 to induce rotation of magnetic rotors 360, 361 and 362 which produces rotation of the rotatably supported turbines 310, 312 and 311 along with their respective magnetic rotors 360, 361 and 362. As is described below in greater detail, it will be noted that the rotation of turbine 310 produces a straight through flow path between input 274 and output 275. In addition, it will be noted that the rotations of turbines 311 and 312 produce a similar straight through blood flow between input 276 and output 277. This straight through flow path is enhanced by the venturi coupling between turbine receptacles 335 and 330 provided by surfaces 321, 322 and 331. The purpose of the venturi coupling is to increase the flow velocity between the pump turbines and further enhance the blood flow between input 276 and output 277. As a result of the straight through blood flow thus produced, areas of stagnation and blood pooling are avoided.

FIG. 12 sets forth a section view of artificial heart 270 showing appropriate connections to the recipient's circulatory system. The blood flow connections are provided by medically approved tube elements and couplers. As described above, artificial heart 270 includes a center housing 271 defining a plurality of turbine receptacles 320, 330 and 335. Receptacles 335 and 330 are aligned coaxially and define cylindrical receptacles. Turbine receptacle 320 is also cylindrical.

Housing 271 further supports a generally cylindrical drive coil array 350 which encircles turbine receptacle 320. Drive coil assembly 350 provides a motor drive coil which is molded into housing 271. Drive coil 350 is coupled to a motor controller such as controller 240 set forth below in FIG. 13. Similarly, housing 271 supports a corresponding drive coil 352 which encircles turbine receptacle 330 and a drive coil 351 which encircles turbine receptacle 335. Drive coils 350, 351 and 352 are substantially identical in fabrication.

Input 274 of pump 270 is coupled to a split coupler 380 which in turn is coupled to the recipient's superior vena cava by a tube 381 and is further coupled to the recipient's inferior vena cava by a tube 382. Similarly, output 275 is coupled to a split coupler 385 which is coupled to the recipient's pulmonary arteries by a pair of tubes 386 and 387. In addition, input 276 is coupled to a split coupler 370 which is coupled to the recipient's pulmonary veins by pair of tubes 371 and 372. Finally, output coupler 277 is coupled to a tube 376 by a coupler 375. Tube 376 is coupled to the recipient's aorta. Output coupler 277 further supports a pair of output transducers 390 and 391 while output 275 supports a pair of output transducers 392 and 393. Transducers 390, 391, 392 and 393 are coupled to the artificial heart controller in the manner set forth below in FIG. 13.

In operation, drive coils 350, 351 and 352 are driven through connecting wires by the controller in the manner set forth below in FIG. 13 to produce rotations of turbines 310, 312 and 311. As turbine 310 is rotated, blood is drawn through coupler 274 into pump 270 from the patient's superior and inferior vena cava through tubes 381 and 382. This blood flows through housing 271 in the direction indicated by arrow 316 and passes through output 275 and thereafter passes through split connector 385 and tubes 386 and 387 to the recipient's lungs. The rotation of turbine 312 draws blood from the patient's lungs through tubes 371 and 372 and split coupler 370 to input 276. Blood flows through turbine 312 in the direction indicated by arrow 315 sand is further pumped by the rotation of turbine 311. The output of turbine 311 passes outwardly through output 277 and coupler 375 to be carried by tube 376 to the recipient's aorta for distribution throughout the recipient's circulatory system.

Thus, as drive coil 350 is energized by the system controller, the rotation of turbine 310 provides a continuous straight through flow of blood from input 274 to output 275. Similarly, energizing of drive coils 351 and 352 rotates turbines 312 and 311 producing a straight through blood flow between input 276 and output 277. This continuous straight through blood flow is in accordance with an important advantage of the present invention artificial heart in that the entire passage through turbines 312 and 311 as well as the venturi coupled between turbines 312 and 311 is a straight through continuous flow without areas of pooling or stagnation being created

In accordance with an important aspect of the present invention, the straight through construction of the pump segment provided by turbines 312 and 311 operates to maintain a blood flow in the direction indicated by arrow 315 from input 276 to output 277 notwithstanding a failure or other interruption of the rotation of either turbine 312 or 311. That is to say, if turbine 312 ceases, for whatever reason to rotate, the pumping action provided by the rotation of turbine 311 continues to maintain blood flow in the direction indicated by arrow 315. Conversely, should turbine 311 for some reason cease rotation, the rotation of turbine 312 continues to provide blood flow in the direction indicated by arrow 315. It will be apparent to those skilled in the art that the combination of straight through blood flow and redundancy of pump turbine segments provides a substantial protection and safety factor for the present invention artificial heart.

Sensors 390 and 391 monitor the output pressure at output 277. Similarly, transducers 392 and 393 monitor the output pressure at output 275. In response to variations of sensed pressure at either of outputs 275 or 277, the system controller (seen in FIG. 13) is able to adjust the operating turbine segments of the artificial heart to compensate for any failures within the quartet of turbines. In addition, it will be apparent to those skilled in the art that the drive signals applied to drive coils 350 through 352 may be modulated or varied to provide a pulsitile output pressure from either or both of outputs 275 and 277. This pulsitile character imparts a more natural blood flow pattern for the recipient and may prove to be advantageous to the recipient.

FIG. 13 sets forth a block diagram of the present invention artificial heart in a typical surrounding environment. In the situation represented in FIG. 13, artificial heart 270, constructed in accordance with the construction of described above, has been implanted within a host patient's body and is operatively coupled in the manner set forth above in FIG. 12 to the host patient's circulatory system. FIG. 13 further shows a microcontroller unit 240 also implanted within the host patient's body. Microcontroller unit 240 is formed of a pair of fully redundant micro controllers 241 and 242. The redundancy of micro controllers 241 and 242, each able to fully support the operation of artificial heart 210, provides a further measure of reliability. Microcontroller unit 240 further includes conventional apparatus (not shown) for communicating to the exterior of the host patient's body in order to provide alarm condition information or other required maintenance of monitoring information to an external unit (not shown). Artificial heart 270 includes a plurality of sensors situated at the respective inputs and outputs of artificial heart 270 which are coupled to redundant microcontrollers 241 and 242. Microcontroller 242 further includes additional sensors supported within artificial heart 270 for monitoring the performance of the servo drive apparatus therein. A pair of battery units 250 and 260 are also implanted within the host patient. Battery unit 250 includes a secondary charging coil 251 coupled to a rectifier 252 which in turn is coupled to a battery 253. Battery unit 250 is coupled to microcontroller 241. Similarly, battery unit 260 includes a charging coil 261 coupled to a rectifier 262 which in turn is coupled to a battery 263. By way of further similarity, battery unit 260 is operatively coupled to microcontroller 242. Thus, microcontroller unit 240, artificial heart 210 and battery units 250 and 260 together with appropriate wire connections therebetween are implanted within a host patient body. For purpose of illustration, FIG. 13 shows body segments 218 and 219 which represent the skin and associated tissues of the host patient body beneath which battery units 250 and 260 are implanted. Preferably, units 250 and 260 are implanted near the host patient's mid section and preferably situated just beneath the patient's skin.

A charging belt 258 suitably configured to be worn by the host patient such as at or near the patient's waist supports a pair of charging units 255 and 256. Charging units 255 and 265 include respective primary charging coils 256 and 266. Coils 256 and 266 are coupled to source of alternating current power such as a conventional electrical outlet 245 via a conventional coupling adapter 246.

In operation, micro controllers 241 and 242 monitor the plurality of sensors within artificial heart 270 and provide suitable operating power and control to the servo drives supported therein. Microcontrollers 241 and 242 utilize batteries 253 and 263 for operative battery supply and for power to energize the servo drive apparatus within artificial heart 210. The operative power stored within batteries 253 and 263 is provided by inductive charging utilizing charging units 255 and 265. Thus, during convenient periods, the host patient utilizes charging belt 258 by coupling to power source 245 while wearing belt 258 such that primary charging coils 256 and 266 are positioned on the outside of body portions 218 and 219 respectively such that general alignment is obtained between primary charging coils 256 and 266 and secondary coils 251 and 261 respectively. Electrical power is then inductively coupled through body portions 218 and 219 to induce alternating current power within secondary coils 251 and 261. Rectifiers 252 and 262 convert the alternating current induced in coils 251 and 261 to a direct current power suitable for charging batteries 253 and 263. In this manner the user is able to replenish the battery energy as required by simply wearing charging belt 258 for a suitable time interval.

Micro controller unit 240 functions using a pair of fully-redundant fully-interconnected micro controllers, each having the complete capability to control and run the entire artificial heart system and it's monitoring and charging functions. Thus, micro controllers 241 and 242 provide inputs for two batteries, inputs for multiple pressure and Hall effect servo sensors and systems capable of monitoring multiple battery charge levels and switch between batteries. The redundancy of micro controllers 241 and 242 includes configuration of the system such that each micro controller “sees” all its own inputs and also “sees” all inputs to the other micro controller. This redundancy includes each micro controller being capable of making compensating performance adjustments to maintain envelope system performance. However, to avoid “hunting” between the redundant micro controllers, it is preferred that small pressure variations of each pump be allowed before adjustment is made.

Micro controller unit 240 further includes communication capability, such as a wireless unit, to call, or text remote locations to indicate system anomalies, failures, operating conditions, battery charge levels and other conditions. In addition, micro controller unit 140 provides the capability to adjust each of micro controllers 241 and 242 based on pressure readings and to set and maintain preset maximum and minimum pressure envelopes.

Micro controller unit 240 also provides the ability of replicating the pulsitile operation characteristic of a normal human heart by introducing pre-programmed increases and decreases of pump speed to create pressure surges and lulls.

While particular embodiments of the invention have been shown and described, it will be obvious to those skilled in the art that changes and modifications may be made without departing from the invention in its broader aspects. Therefore, the aim in the appended claims is to cover all such changes and modifications as fall within the true spirit and scope of the invention.

That which is claimed is:

1. An artificial heart comprising:
a housing having a first input, a first output, a first blood flow channel between said first input and said first output and a second input, a second output and a second blood flow channel between said second input and said second output;
a first turbine pump operative, within said first blood flow channel, to flow blood from said first input to said first output;
a second turbine pump operative, within said first blood flow channel, to flow blood from said first input to said first output; and
a third turbine pump operative, within said second blood flow channel, to flow blood from said second input to said second output.

2. The artificial heart set forth in claim 1 wherein said first and said second turbine pumps are arranged in series blood flow within said first blood flow channel and said third turbine pump is within said second blood flow channel.

3. The artificial heart set forth in claim 2 wherein said housing defines a plurality of turbine receptacles and wherein said first, second, and third turbine pumps each include:
a respective turbine receptacle:
a turbine rotatably supported within said turbine receptacle:
a magnetic rotor rotatable with and supported by said turbine and
a drive coil supported within said housing and encircling said turbine receptacle and said turbine and said magnetic rotor.

4. The artificial heart set forth in claim 3 wherein said housing is molded and wherein said drive coils are molded into said housing.

5. The artificial heart set forth in claim 4 wherein said turbines each include:
a turbine shaft having bearings at opposed ends thereof; and
a helical turbine blade supported upon and extending from said turbine shaft defining an outer edge.

6. The artificial heart set forth in claim 5 wherein said magnetic rotor is generally rotors are cylindrical and define a respective interior surface and wherein each said outer edge of said each turbine blade receives said respective interior surface to join said magnetic rotor to said turbine blade.

7. An artificial heart comprising:
a housing having a first input, a first input turbine receptacle, a first output turbine receptacle, a first output and a first coupling passage between said first input turbine receptacle and said first output turbine receptacle, said first input, said first input turbine receptacle, said first output turbine receptacle, said first output and said first coupling passage being generally coaxial and said housing further having a second input, a second input turbine receptacle, a second output and a second coupling passage between said second input turbine receptacle and said second output, said second input, said second input turbine receptacle, said second output and said second coupling passage being generally coaxial;
a first turbine pump supported within said first input turbine receptacle operative to flow blood from said first input through said first coupling passage to said first output turbine receptacle;
a second turbine pump supported within said first output turbine receptacle operative to flow blood from said first coupling passage to said first output; and
a third turbine pump supported within said second input turbine receptacle operative to flow blood from said second input through said second coupling passage to said second output turbine receptacle.

8. The artificial heart set forth in claim 7 wherein said first, second and third turbine pumps supported within a respective turbine receptacle each include:
a turbine rotatably supported within its respective turbine receptacle:
a magnetic rotor rotatable with and supported by said turbine and
a drive coil supported within said housing and encircling said respective turbine receptacle and said turbine and said magnetic rotor.

9. The artificial heart set forth in claim 8 wherein said housing is molded and wherein said drive coils are molded into said housing.

10. The artificial heart set forth in claim 9 wherein said turbines each include:
a turbine shaft having bearings at opposed ends thereof; and
a helical turbine blade supported upon and extending from said turbine shaft defining an outer edge.

11. The artificial heart set forth in claim 10 wherein said magnetic rotor is generally rotors are cylindrical and define a respective interior surface and wherein each said outer edge of said each turbine blade receives said respective interior surface to join said magnetic rotor to said turbine blade.

12. An artificial heart comprising:
a housing having a first input, a first input turbine receptacle, a first output turbine receptacle, a first output and a first coupling passage between said first input turbine receptacle and said first output turbine receptacle, said first coupling passage being curved, said housing further having a second input, a second input turbine receptacle, a second output and a second coupling passage between said second input turbine receptacle and said second output, said second coupling passage being curved;
a first turbine pump supported within said first input turbine receptacle operative to flow blood from said first input through said first coupling passage to said first output turbine receptacle;
a second turbine pump supported within said first output turbine receptacle operative to flow blood from said first coupling passage to said first output; and
a third turbine pump supported within said second input turbine receptacle operative to flow blood from said second input through said second coupling passage to said second output turbine receptacle.
